(19) United States
(12) Patent Application Publication
(10) Pub. No.: US 2024/0285175 A1
(43) Pub. Date: Aug. 29, 2024

(54) DEVICE FOR ASCERTAINING THE PHYSIOLOGICAL STATE OF BABIES AND SMALL CHILDREN

(71) Applicant: LEEVI Health GmbH, Simmern (DE)

(72) Inventors: Nadine WUNDERLICH, Simmern (DE); Bjoern WUNDERLICH, Simmern (DE)

(73) Assignee: LEEVI Health GmbH, Simmern (DE)

(21) Appl. No.: 18/559,929

(22) PCT Filed: May 27, 2022
(86) PCT No.: PCT/EP2022/064386

§ 371 Date: Nov. 9, 2023

(30) Foreign Application Priority Data
May 28, 2021 (EP) 21176722.3

(51) Int. Cl. A61B 5/0205; A61B 5/00; A61B 5/024; A61B 5/08; A61B 5/145; A61B 5/1455

(52) U.S. Cl. CPC A61B 5/02055; A61B 5/002; A61B 5/0022; A61B 5/14532; A61B 5/14551; A61B 5/681; A61B 5/6843; A61B 5/7267; A61B 5/7275; A61B 5/746; A61B 5/02416; A61B 5/02438; A61B 5/0816; A61B 2503/04; A61B 2503/06; A61B 2560/0252; A61B 2562/0247; A61B 2562/029

(57) ABSTRACT

The invention relates to a portable device which is designed to be carried on the body of a child, said child being a baby or a small child. The device includes one or more sensors for detecting multiple vital parameters of the child, wherein the vital parameters at least include the heart rate, oxygen saturation, and respiratory rate; analysis software which is configured so as to predict at least one current or future physiological state of the child as a function of the heart rate, oxygen saturation, and respiratory rate measured by the sensors; and an interface for transmitting the prediction result relating to the at least one physiological state to a mobile telecommunication device of a user and/or to a server computer system.

### FIELD

The present invention relates to a device for ascertaining the physiological state of a baby or small child, which is carried on the body of the child and which contains a plurality of sensors.

### PRIOR ART

Various devices and sensor systems for monitoring the vital parameters of newborns and small children are known in the prior art. In a decidedly medical context, e.g. in a neonatal ward or intensive care unit of a hospital, the devices are often characterised by high precision in detecting life-threatening physiological states in particular. However, the sensor systems used for this purpose are often very expensive, and it takes time, experience and often a large number of complex and large devices to correctly attach the sensors to the child's body. These devices, configured for a neonatal unit, are therefore not suitable for monitoring the physiological states of children in a home environment by the child's parents.

In the home environment, there are now a number of ways for parents to monitor at least some of their children's physiological states using comparatively simple means.

Patent application US 2016324466 A1 describes a method, device and system for localising and monitoring environmental risk factors for sudden infant death syndrome (SIDS). The device is used to monitor the sleep environment of newborns and infants at home by a parent or other caregiver. The device is placed near the infant's face and monitors, e.g., the CO₂ content of the exhaled air and the lying position of the child. In particular, the sleeping position and possible covering of the head by the bedding is considered a risk factor, as this may block the airways and impair breathing. Blood parameter values are not monitored.

Patent application US 2018000405 A1 discloses a system and methods for health monitoring. The system detects various vital parameters of the mother in the puerperium as well as various parameters of the newborn, such as foetal heart rate and oxygenation. However, it is not described that machine learning methods are used to predict physiological parameters, in particular to detect an increased risk of sudden infant death syndrome or to detect a feeling of hunger.

Patent application US 2020/0060590 A1 describes a baby monitor consisting of a sensor unit and a receiver unit. The sensor unit contains various sensors, a processing unit and a transmitter unit. The processing unit processes the raw data measured by the sensors, in particular it formats said data. The transmitter unit sends the formatted data to the receiver unit. The sensor unit is attached to the baby's foot and contains sensors for measuring the heart rate, the oxygen content of the blood and the movement measurement. The heart rate and oxygen content are measured using pulse oximetry. The receiver unit (but not the sensor unit) analyses the data received and triggers an alarm if necessary.

Many devices used in the prior art for monitoring vital parameters in the home environment have various problems. They often contain only a few sensors, as a larger number of sensors may often not be easily integrated into clothing or accessories that may be worn by babies and small children, as there is little space or surface available for attaching the sensors. The small number of sensors also often means that the database is not very comprehensive and the predictions based on it are of poor quality. Adding more sensors would also often make the device significantly more expensive.

Another problem with some prior-art devices is that the measurement data alone is often only of limited use to the user. An altered respiratory rate or a reduced oxygen concentration may have various causes, meaning that these values alone do not allow parents to recognise whether there is a problem.

### SUMMARY

The object of the invention is to provide an improved device for recognising the physiological states of a baby or small child, which does not exhibit the above-mentioned problems or exhibits them to a lesser extent.

The objects of the invention are achieved in each case by the features of the independent claims. Embodiments of the invention are described in the dependent claims. The embodiments listed below are freely combinable with one another, provided that they are not mutually exclusive. In one aspect, the invention relates to a portable device. The portable device is configured to be carried on the body of a child (“wearable”). The child is a baby or small child. The device comprises:
one or more sensors for detecting a plurality of vital parameters of the child, the vital parameters comprising at least heart rate, oxygen saturation, and respiratory rate; and
evaluation software configured to predict at least one current or future physiological state of the child as a function of the heart rate, oxygen saturation, and respiratory rate measured by the sensors; and
an interface for transmitting the prediction result relating to the at least one physiological state to a mobile telecommunication device of a user and/or to a server-computer system.

This may be advantageous because the above-mentioned parameters have been shown to be particularly predictive of a variety of relevant physiological states, including in particular physiological states in which there is an increased risk of sudden infant death syndrome. The evaluation of said parameters is advantageous because it may be carried out by means of sensors which may be attached directly to the body, so that the measured values obtained are less susceptible to relative movements of the child in relation to external sensors and less susceptible to various external influencing factors. The applicant has observed that the three parameters mentioned above are highly predictive of an increased risk of sudden infant death syndrome. Although the accuracy may be further increased by taking other parameters into account, a sufficiently accurate prediction quality is already possible on the basis of the three parameters mentioned above in order to reliably warn of risk situations relating to SIDS on the one hand, yet not trigger so many false alarms that parents would feel compelled to deactivate the function on the other.

External sensors such as external cameras or microphones for monitoring the child's position or breathing have the disadvantage that the child may move out of the sensor range, meaning that critical situations may no longer be detected. Another disadvantage is that setting up the sensor environment is so complex that in many situations, e.g. when travelling on holiday or when the child is lying on the sofa in the living room and not in the cot, the sensor environment is not available at all. This creates gaps in protection. The fact that the device is configured as a “wearable” with the corresponding sensor technology and analysis software means that there is no need to set up the sensor environment and it is also impossible for the child to move away from the area monitored by the external sensors.

The three minimum vital parameters detected are also comparatively less susceptible to faults: an increased CO₂ concentration in the outside air is not necessarily an indication that the child has breathing problems. It is possible that the room air is generally depleted. The evaluation of the acoustic signal from external microphones with regard to breathing noises may also be disturbed by background noise, such as renovation work, or by a blanket sliding in front of the microphone. These problems do not exist with the three vital parameters mentioned above.

In a further advantageous aspect, it is possible to detect all three parameters via the same sensor or to derive them from the raw data of a single sensor, e.g. if a photoplethysmographic sensor, referred to here as a PPG sensor, is used.

Embodiments of the invention may make it possible to predict the occurrence of problematic physiological states before they actually occur, so that parents or caregivers may take countermeasures in good time.

In a further advantageous aspect, the device comprises at least the sensor or sensors required to detect or derive said three vital parameters. Optionally, the wearable device may include a number of other sensors for further vital parameters and/or one or more environmental parameters. This means that the child does not have to be wired. Putting on or “donning” the device is sufficient to bring the large number of sensors into contact with the child's body. This means that the child's natural movements are not hindered by cables and it is ensured that there are no gaps in protection when the child is taken out of a “monitored” environment temporarily or while travelling.

Unlike in systems that measure the child's vital parameters using external sensors, there is no risk of the measurements being falsified by the child's movements relative to the external measuring unit. As the device is attached to the child's body, it also follows the child's movements.

In a further advantageous aspect, the predicted physiological state of the child is output or transmitted to the telecommunication device as a result of the prediction. The telecommunication device may be, for example, a smartphone of the parent or caregiver. The user therefore does not have to interpret individual physiological parameters, but is informed directly about the child's probable physiological state.

Additionally or alternatively, some of the data collected or derived from the wearable device, the prediction results or intermediate prediction results may also be transmitted to a server-computer system via a network. For example, the server-computer system may further process the data received from the wearable device. Further processing may, for example, consist of carrying out more complex, computationally elaborate analyses with the data and/or storing the raw data in a database. Further processing may include combining the data from the wearable device with data from other external sensors to obtain a final prediction result regarding the at least one physiological state and storing and/or sending this final prediction result to the parent's telecommunication device over the network.

In a further advantageous aspect, data processing takes place directly on the device, at least with regard to those physiological states that require immediate intervention by the caregivers.

According to embodiments, the prediction result, optionally supplemented by some of the parameter values (raw data) detected by the sensors, is only sent to the telecommunication device if a current, critical physiological state has been calculated or an acutely critical vital parameter value or environmental parameter value has been detected or if the caregiver has explicitly requested data transmission via the telecommunication device (using the pull function).

This reduces data traffic over the network and may also extend the battery life, as preparing the data for transmission and the transmission itself requires computing power and therefore energy. Operating the radio module, especially in “normal-radiation” operating mode, also requires energy.

### Predicting an Increased Risk of Sudden Infant Death Syndrome

According to embodiments, the at least one physiological state is a state of increased risk of sudden infant death syndrome. The evaluation software is configured to use at least the heart rate, the oxygen saturation, and the respiratory rate as input to predict the acute or future presence of an increased risk of sudden infant death syndrome.

For example, the evaluation software may include a predictive model for predicting sudden infant death syndrome, referred to here as a “SIDS model”. A SIDS model is understood here as a predictive model for predicting the increased risk of sudden infant death syndrome. The SIDS model is configured to use at least the heart rate, oxygen saturation, and respiratory rate, and optionally some other vital and environmental parameters as input to predict the presence of an increased risk of sudden infant death syndrome.

The SIDS model is preferably a model based on machine learning, in particular a neural network. However, alternative embodiments are also possible, such as a rule-based system.

The use of said parameters has the advantage that an increased risk of sudden infant death syndrome (SIDS) may be recognised with greater sensitivity and specificity than was previously possible in the segment of devices for home use. High sensitivity is particularly important here, as sudden infant death syndrome is one of the most common causes of death in babies and small children. However, high specificity is also very important, as every false alarm is very stressful for parents on the one hand, and on the other, an excessively high false alarm rate also harbours the risk that an alarm will be ignored in an emergency.

The increased quality of the prediction is due in particular to the combined evaluation of the aforementioned parameters heart rate, oxygen saturation and respiratory rate.

The combined evaluation of heart rate, oxygen saturation and respiratory rate allows an increased risk of sudden infant death syndrome to be recognised earlier, before respiratory arrest or abnormal breathing patterns occur. Parents may thus be alerted earlier. Valuable time is gained for the prevention of sudden infant death syndrome.

Abnormalities in breathing (apnoea (breathing interruptions), irregular breathing rate) indicate an increased risk of SIDS even before hypoxaemia occurs. As the pathophysiology progresses, bradycardia (lower heart rate) may also occur. Finally, there is a sharp drop in the oxygen concentration in the blood (hypoxaemia) and the child gasps for air. Normally, the autonomic nervous system would recognise the hypoxemia and take countermeasures. In sudden infant death syndrome, however, this counter-reaction may fail to materialise for reasons that are, as yet, unknown. A possible cause of this is thought to be a lack of maturity of the autonomic nervous system. This is followed by a further drop in oxygen levels and SIDS.

According to embodiments of the invention, the evaluation software is configured to calculate an increased risk for the current or future occurrence of SIDS and to generate various alarm messages (coded in different colours according to urgency, for example) and to output them directly or indirectly (via the server-computer system) to the caregiver's telecommunication device:
In the event of irregular breathing patterns (apnoea<DURATION) and otherwise normal heart rate and oxygen concentration: Warning: Consult paediatrician!
In the event of irregular breathing patterns (apnoea>DURATION) and otherwise normal heart rate and oxygen concentration: output of an acute alarm, level 1, orange;
In the event of irregular breathing patterns (apnoea>DURATION)+hypoxia+normal heart rate: output of an acute alarm, level 1, orange;
In the event of irregular breathing patterns (apnoea>DURATION)+hypoxia+bradycardia: Output of an acute alarm, level 2, red.

The parameter DURATION is preferably a value in the range of 12 to 19 seconds, in particular a value of 14 to 17 seconds, e.g. 15 seconds, 16 seconds or 16.5 seconds.

The combined evaluation of the parameters enables an earlier and more reliable warning. Children with an increased risk of SIDS may be recognised at an early stage so that a corresponding medical examination may be recommended to the caregivers at an early stage. As the detected vital parameters (respiratory rate, heart rate, oxygen saturation) are preferably stored in the wearable device, the caregiver's telecommunication device or the server-computer system, parents may provide the doctor with meaningful long-term data on the child's vital parameters to facilitate diagnosis. In addition, the wearable device may detect exogenous stressors such as thermal stress (due to prone position or excessively warm ambient temperature), obstruction due to prone position, covering of the face due to prone position (blankets/pillows) so that caregivers may intervene immediately.

According to a further embodiment, the wearable device includes a temperature sensor for measuring the temperature of the child's skin. Preferably, the wearable device also includes a temperature sensor for measuring the ambient temperature.

The body temperature in combination with the ambient temperature may be used by the evaluation software to increase the accuracy of the prediction of the increased risk of SIDS. The combination of body temperature and ambient temperature allows at least an approximate derivation of the core body temperature. A strongly elevated core body temperature may, for example, indicate heat accumulation, which may increase the risk of SIDS. By analysing the skin temperature in combination with the ambient temperature, the quality of the prediction regarding the presence of an increased risk of SIDS may be increased further still.

According to some embodiments, the device also contains an air moisture sensor, the measured values of which are also taken into account in said prediction. If the air moisture is high, the child is even less able to compensate for heat build-up by increased perspiration. By taking these risk factors into account (increased body temperature, possibly in combination with the ambient temperature and optionally also the moisture level of the air surrounding the device), the prediction quality is increased.

According to some embodiments, the evaluation software is configured not only to generate a prediction result stating whether there is an increased risk of SIDS, but also to output the relevant risk parameters themselves (e.g. reduced oxygen concentration in the blood, altered heart or respiratory rate, excessive body or ambient temperature, etc.). This gives parents the opportunity to counteract the relevant risk factors in a targeted manner. For example, the child's lying position may be changed, a blanket removed or the room temperature lowered by opening windows.

Preferably, the thermometer for measuring the local skin temperature of the child is located on the support of the device, the thermometer preferably having direct skin contact.

According to embodiments, the device has a thermometer for measuring a local skin temperature of the child and a thermometer for measuring the ambient temperature.

For example, the skin temperature sensor may be attached to the inside of a device in the form of a ribbon, which is in direct contact with the child's skin. The ambient temperature sensor may be attached to the outside of the ribbon. However, according to some embodiments, the external temperature sensor may also be formed as an external sensor that transmits the ambient temperature data to the wearable device and/or the server-computer system via a base station.

This may be advantageous as the prediction quality is further increased. For example, an increased body temperature measured at the skin is less problematic if the ambient temperature is high, as the latter also directly influences the skin temperature. However, an increased skin temperature at low outside temperatures is a clear sign of physiological overheating, e.g. due to too many blankets on the child.

According to embodiments, the evaluation software derives the core body temperature or changes in the core body temperature from the measured skin temperature of the child. The body temperature derived in this way is then passed on together with the ambient temperature as input to the analysis software to predict an increased risk of SIDS.

Methods for deriving core body temperature from skin temperature are known and are described, for example, for adult men in the following publication: Eggenberger P, et al: “Prediction of Core Body Temperature Based on Skin Temperature”, Heat Flux, and Heart Rate Under Different Exercise and Clothing Conditions in the Heat in Young Adult Males. Front Physiol. 2018; 9:1780. Published 2018 Dec. 10. doi:10.3389/fphys.2018.01780. A corresponding dataset may also be generated for children, in which skin temperature, ambient temperature and core body temperature measured simultaneously under different conditions are linked. By performing, for example, a regression analysis on this data, a function specified as a formula or equation or a predictive model based on machine learning may be generated, which is able to derive the core body temperature from the skin temperature. The applicant has observed that the use of a derived body temperature instead of the directly measured skin temperature may further increase the quality of the prediction of an increased risk of sudden infant death syndrome, as the core body temperature is less influenced by environmental disturbance parameters and correlates more strongly with SIDS risks than the skin temperature.

For example, by using the derived core body temperature instead of the skin temperature, the effect of centralisation of the blood during fever and a temporal pattern of change in core body temperature may be detected.

According to embodiments, the evaluation software is further configured to recognise the presence of and/or predict the future presence of fever. The evaluation software may use a profile of the change in the derived core body temperature and optionally one or more other parameters as input to predict the current or future presence of fever. If fever is predicted, a corresponding message (fever alarm) is issued directly by the device and/or transmitted to the telecommunication device via the interface.

Thus, by analysing all the data (e.g. heart rate, respiratory rate, blood oxygen concentration, derived core body temperature and optionally also ambient temperature), a better prediction quality may be achieved with regard to sudden infant death syndrome.

According to embodiments, the sensors comprise a photoplethysmographic sensor, referred to here as a PPG sensor. The evaluation software is configured to derive both the heart rate and the oxygen saturation and respiratory rate of the child from the signals detected by the PPG sensor and to make them available as input to the evaluation software for predicting a current or future increased risk of sudden infant death syndrome.

The use of a PPG sensor to derive the above-mentioned vital parameters from the PPG signals may be advantageous for several reasons: firstly, it saves space so that additional sensors may easily be accommodated in the device. In addition, the device may be produced more cheaply, it is lighter and also less susceptible to faults, as it has a smaller number of sensors than would be necessary if a separate sensor had to be installed for each of said parameters. The applicant has observed that the data generated by current PPG sensors contains sufficient information to derive said parameters.

For example, the PPG sensor may be a photoplethysmographic probe with a light-emitting element and a light-detecting element. The light-emitting element may, for example, consist of a laser or a combination of several lasers. The spectrum and the intensity of the reflected light of the respective lasers provide information about the amount of blood that is pumped through the vein system located near the PPG sensor at a certain point in time and thus also allow the heart rate to be derived from the raw data. Since inhalation and exhalation have an influence on the arterial blood flow, it is possible to derive the respiratory rate from the PPG signal. In addition to heart rate and oxygen saturation, abnormalities in respiratory rate are an important prognostic factor for the risk of SIDS.

The light signals detected by the PPG make it possible to recognise fluctuations in the amount of blood transported per unit of time. As these fluctuations are influenced by the heartbeat and respiration, among other things, the analysis software may also recognise the heartbeat and respiratory rate from the PPG sensor data.

For example, the heart rate may be detected or calculated using a PPG sensor as follows: the PPG sensor contains one or more light sources, e.g. LEDs of certain wavelengths, which emit light that passes through the skin and (among other things) hits blood vessels. The light is absorbed, scattered and reflected by the tissue and the vessels contained thereby. A photodetector measures the intensity of the transmitted or reflected light.

As the absorption properties of blood and other tissue components differ, changes in the volume of the blood vessels may be analysed in the plethysmogram. The wave-shaped plethysmogram is composed of the “direct current” (DC) and “alternating current” (AC) components. The DC component depends mainly on the structure of the tissue and on the mean blood volume of the arterial and venous blood. Changes in venous capacitance are detectable as changes in the DC component. The AC component reflects the change in volume during systole and diastole of the heart. The heart rate may be determined on the basis of this pulsatility.

Preferably, the PPG sensor is used to detect not only the heart rate, but also the respiratory rate or to derive it from the raw data, as breathing and the cardiovascular system influence each other.

Inspiration and expiration lead to arterial and venous blood volume fluctuations due to changes in intrathoracic pressure. Intrathoracic negative pressure during inspiration causes the pressure in the veins to fall and the venous inflow to the heart to increase. Systolic blood pressure in particular falls and the heart rate increases. The opposite effect occurs during expiration.

These respiration-dependent fluctuations in blood pressure and heart rate lead to fluctuations in blood volume and thus to fluctuations in the intensity measured at the photodetector.

This means that the PPG sensor may also be used to determine the child's breathing rate.

The derivation of the respiratory rate from a PPG signal may be performed, for example, as described in Nilsson LM. Respiration signals from photoplethysmography. Anesth Analg. 2013 October; 117(4):859-65. doi: 10.1213/ANE.0b013e31828098b2. Epub 2013 Feb. 28. PMID: 23449854. The heart rate may also be derived from the PPG data in a similar way.

The values may be influenced by other movements of the child, and so movement is a possible source of error. However, by using filters, fluctuations in blood flow caused by movement of the child (other than breathing movement!) may be recognised and filtered out. In addition, according to embodiments of the invention, the PPG signal is used to determine blood parameters of the child, in particular oxygen saturation and preferably other blood parameters, by means of which the quality/accuracy of the prediction of an increased risk of sudden infant death syndrome may be improved and/or by means of which other physiological states may be predicted or recognised.

The other blood parameters that may be used to improve the quality/accuracy of the prediction of an increased risk of sudden infant death syndrome (i.e. which serve as “control blood parameters”) are, in particular, blood parameters that do not correlate with the blood oxygen concentration or correlate negatively or correlate positively with blood oxygen in a known and non-linear way.

A blood parameter is a measured value that results from a certain property of the blood, e.g. the concentration of a certain molecule in the blood.

A blood parameter that correlates negatively with the blood oxygen concentration is, for example, a blood parameter that decreases in strength when the blood oxygen concentration rises and increases in strength when the blood oxygen concentration falls, e.g. the CO₂ concentration in the blood.

A blood parameter that is not correlated with the blood oxygen concentration is thus, e.g., a blood parameter of which the strength is at least approximately independent of the blood oxygen concentration. For example, the concentration of carboxyhaemoglobin depends substantially on the carbon monoxide concentration in the air, not on the oxygen concentration, as carbon monoxide displaces oxygen from haem. In general, however, other blood parameters which are derived from a blood component that correlates positively with the oxygen concentration in a known non-linear manner, e.g. according to an exponential or polynomial relation, may also be used as control blood parameters. For example, if a 30% drop in blood oxygen concentration is detected, and a particular blood component that is known to increase or decrease as a function of oxygen concentration, e.g. if the measured or derived concentration of this blood component also falls by 30% in the same way as the oxygen concentration, a measurement error must be assumed, e.g. because the PPG sensor from the raw data of which both the oxygen concentration and the control parameter are derived has shifted. However, if the blood component in question decreases by 90% when the oxygen concentration drops by 30%, it may be assumed that there is actually a drop in the oxygen concentration in the blood, as an error in the sensor technology, e.g. due to a lack of contact, is in most cases incorporated linearly and equally into the measurements of all measured values of these sensors.

According to some embodiments, the one or more sensors comprise a sensor for detecting at least one blood parameter of the child, wherein the blood parameter is, for example, a CO₂ concentration in the blood, a methaemoglobin concentration and/or a carboxyhaemoglobin concentration in the blood of the child.

For example, the sensor for detecting the blood parameter may be the PPG sensor, which is already used to detect the blood oxygen content, respiratory rate and heartbeat. This is advantageous because no additional sensor is required and the same sensor that already measures the oxygen concentration in the blood or derives it from the raw data may be used.

The evaluation software is configured to use the at least one blood parameter (CO₂ concentration, methaemoglobin concentration and/or a carboxyhaemoglobin concentration in the blood) as an additional input parameter in order to reduce the false positive rate of the prediction of the increased risk of sudden infant death syndrome. In addition or as an alternative to said three minimum vital parameters: heart rate, respiratory rate and blood oxygen concentration, one or more of the control blood parameters constituted by CO₂ concentration, methaemoglobin and/or carboxyhaemoglobin concentration in the blood may also be used as control parameters in the prediction of an increased risk of SIDS.

Carboxyhaemoglobin (HbCO) is formed by the reversible binding of carbon monoxide (CO) to the iron ion of the haem group. Carbon monoxide binds to haemoglobin in the same places as oxygen, but about 200 times more strongly. This means that HbCO may bind almost no oxygen. As carbon monoxide is not present in normal room air, or not in significant quantities, it may be assumed that the CO concentration remains constant under normal conditions. If, in addition to an oxygen concentration that is too low, a carboxyhaemoglobin concentration that is too low is also measured, it may be assumed that there is a measurement error. If, on the other hand, the carboxyhaemoglobin value remains constant, the analysis software may assume that the oxygen concentration is actually low.

Methemoglobin is a form of haemoglobin that may also no longer transport oxygen. It is produced by oxidation of the bivalent iron (Fe2+) in the haem group to trivalent iron (Fe3+). The physiological concentration of methaemoglobin in the blood is low at less than 1%, but the concentration may be increased by certain chemical compounds.

Like carboxyhaemoglobin, methaemoglobin is a blood parameter that is generally present in the blood in a constant concentration and may therefore be used as a control parameter. According to embodiments, the methaemoglobin content is transferred to the evaluation software as a further input parameter (“control parameter”) so that said software may recognise whether there is a measurement error or the oxygen concentration is actually too low by comparing the entered blood oxygen concentration with this or other control values.

Measuring the concentration of said substances in the blood or deriving this concentration from the PPG sensor signals may be advantageous because said blood parameters may be used as control parameters to avoid false positive predictions and false alarms. If, for example, the oxygen concentration measured is too low, the evaluation software may use one or more of said control blood parameters to recognise whether there really is an increased risk of sudden infant death syndrome or whether a measurement error is the cause of the low oxygen concentration.

Such measurement errors may occur due to movement of the device when the child moves. If the oxygen concentration in the blood is significantly reduced, but at the same time the CO₂ concentration in the blood is within the normal range or even increased, it is likely that the oxygen concentration in the blood is actually too low. If the concentration of CO₂ (or another control substance such as carboxyhaemoglobin or methaemoglobin) in the blood is also low, it is likely that the measurement is incorrect. CO₂ as a control substance should rise in the event of a real reduction in oxygen saturation, in the event of a false alarm CO₂ would also fall as O₂.

The fact that the evaluation software uses and takes into account additional control parameters as input parameters means that false alarms may be avoided, which is particularly important in the context of recognising a life-threatening physiological state.

According to embodiments, the device comprises at least one sensor for determining at least one further vital parameter and/or environmental parameter.

In addition or as an alternative to the sensor for the environmental parameter, the device may also include an interface for receiving the further vital parameter(s) and/or environmental parameter(s) from one or more external sensors. For example, data may also be determined by a further component that is installed in the room in which the child is located and transmitted to the device on the child's body and/or to the server-computer system. The at least one further environmental parameter may be, in particular, the CO₂ concentration of the ambient air or the air moisture. The at least one further vital parameter may include video data or movement data that characterise the movement activity of the child. Acoustic data from a microphone built into the wearable device or configured as an external sensor may also be transmitted to the wearable device and/or the server-computer system and used as further input data when predicting an increased risk of SIDS.

The evaluation software is configured to use the at least one further vital parameter and/or environmental parameter as an additional input parameter in order to predict the presence of an increased risk of sudden infant death syndrome.

For example, one or more sensors may be attached to the mattress of a child's bed or as a sticker on the duvet cover, pyjamas or sleeping bag. The external sensors may be motion sensors or pressure sensors, for example, which measure the movement of the chest during sleep. Additionally or alternatively, a motion sensor, e.g. a gyroscope, may also be built into the wearable device. According to embodiments, this movement data is also used as a further input parameter by the evaluation software to reduce the false-positive rate of the predictions and improve the quality of the prediction: in the case of a child whose chest is moving, this may be an indication that breathing is functioning normally and that a reduced oxygen concentration in the blood is probably due to a measurement error. In particular, the microphone and/or video camera and the measurement data thereof may be used to reduce the false-positive rate of SIDS prediction.

According to a further embodiment, an external or internal sensor measures the CO₂ concentration of the ambient air. This parameter may be used as a further input parameter by the evaluation software in order to increase the accuracy of the prediction. A high CO₂ content in the ambient air is an indication that the ambient air is depleted. If the CO₂ value is too high, this indicates unfavourable ambient conditions that may increase the risk of sudden infant death syndrome.

According to another embodiment, an external or device-internal acoustic signal sensor (a microphone) detects sounds of the child and of the environment (since a microphone detects both the sounds of the environment and those of the child, it is both an environmental sensor and a vital parameter sensor). The detected acoustic signal may be used as another input parameter to increase the prediction accuracy. If the child is crying, there may be other problems, but not a lack of oxygen or an increased risk of sudden infant death syndrome.

Additionally or alternatively, the movement data from the acceleration sensor of the wearable device and/or video data from an external camera pointing at the child may be used to detect the child's movement or movement patterns and make this movement data available to the analysis software as input data. A child that moves a lot may be assumed not to be at increased risk of sudden infant death syndrome.

According to one embodiment, the video camera is an infrared camera. This is particularly advantageous because the images from an IR camera enable image analysis software to recognise whether a child's face, which is usually clearly visible in an IR camera against the background of heat-insulating clothing or blankets, is pointing upwards or downwards and thus indicates whether the child is in a prone or supine position. A prone position increases the risk of sudden infant death syndrome as the child breathes into the mattress and/or because the temperature exchange may be restricted.

According to one embodiment, the prediction results of the wearable device regarding the presence of an increased risk of sudden infant death syndrome are first transmitted to the server-computer system as an intermediate result. The server-computer system is operatively linked to the IR camera via a network, e.g. directly or indirectly via a base station. The server-computer system receives the IR images of the child from the external camera and analyses them using image analysis software. The image analysis is comparatively computationally complex, so this analysis is preferably carried out on the server and not on the wearable device, which has only limited computing power. The result of the image analysis is whether the child is in the prone or supine position. The server-computer system is configured to calculate a final result regarding the presence of an increased risk of sudden infant death syndrome from the intermediate result of the wearable device and the result of the image analysis and to send it to the telecommunication device of the caregiver.

### Predicting Feelings of Hunger

According to embodiments of the invention, at least one of the sensors for the vital parameters is configured to determine the child's blood sugar concentration in a non-invasive manner. The evaluation software is configured to recognise a current or future feeling of hunger in the child. The evaluation software is configured to use at least the measured blood sugar concentration as input in order to predict the current or future presence of a feeling of hunger and/or the time of occurrence of the feeling of hunger. The physiological state to be predicted is therefore a state in which the child is hungry. For example, a feeling of hunger is predicted if the current or future blood sugar level is below a predefined threshold value. According to other embodiments, the prediction of the feeling of hunger may also be based on more complex algorithms that take into account other vital parameters of the child or environmental parameters in addition to the blood sugar concentration. For example, the ambient temperature and/or current or previous movement patterns or movement activity of the child may also have an influence on the current or future presence of a feeling of hunger. For example, higher ambient temperatures often reduce the feeling of hunger, increased physical activity may temporarily reduce the feeling of hunger, but if physical activity is reduced following a longer period of physical activity, the feeling of hunger may increase. In addition to a simple prediction algorithm based on a limit value for the blood sugar concentration, other prediction algorithms that take other parameters (vital parameters, environmental parameters) into account may also be used to predict the feeling of hunger according to other embodiments. The prediction algorithm may be a rule-based “if-then” prediction regarding the exceeding of limit values for the one or more parameters, or a predictive model that was generated in a machine learning step. The predictive model may be a neural network, for example. Combinations are also possible, e.g. the prediction by means of a trained network that the blood glucose concentration will be below a certain threshold value at a certain point in time, which is interpreted as the presence of a feeling of hunger at this point in time.

For example, the evaluation software may be configured to calculate the child's current and/or future blood sugar level as a function of the flow properties of the blood and to predict a current or future feeling of hunger on the basis of the calculated blood sugar level.

The flow properties of the blood depend, among other things, on the blood sugar level. The blood sugar level is approximately proportional to the viscosity of the blood and inversely proportional to the flow rate. The evaluation software may, for example, contain a convolutional neural network that may derive the blood sugar level from the PPG signal. For example, the derivation may be performed using said networks as described in S. Hossain, B. Debnath, S. Biswas, M. J. Al-Hossain, A. Anika and S. K. Zaman Navid, “Estimation of Blood Glucose from PPG Signal Using Convolutional Neural Network,” 2019 IEEE International Conference on Biomedical Engineering, Computer and Information Technology for Health (BECITHCON), 2019, pp. 53-58, doi: 10.1109/BECITHCON48839.2019.9063187.

Alternatively, the blood sugar value may also be derived from the PPG sensor signal according to a method as described in Delbeck S, et al: “Non-invasive monitoring of blood sugar using optical methods for skin spectroscopy-opportunities and recent advances”, Anal Bioanal Chem. 2019 January;411(1):63-77. doi: 10.1007/s00216-018-1395-x. Epub 2018 Oct. 3. PMID: 30283998 is described: the PPG sensor performs pulsed measurements in the short-wave near-infrared spectral range with LEDs at 935, 950 and 1070 rnm. The glucose concentration was predicted using an artificial neural network (ANN) after pre-processing of the time-dependent signals by an adaptive noise reduction filter (Adaline) based on the neural network. After training the neural network, the network is used to predict the blood sugar level on the basis of the spectral data collected by the PPG sensor.

According to embodiments, the evaluation software may include or be operatively coupled to a further neural network or another prediction algorithm, wherein the further neural network or the other prediction algorithm is configured to predict the current or future feeling of hunger as a function of the calculated glucose concentration in the child's blood. For example, the other neural network may also be a convolutional neural network. Correct detection or early prediction of a child's hunger may be beneficial and important for parents for many reasons: Children are not yet able to express themselves verbally, so it is often not possible for parents to recognise whether a child's crying is caused by hunger, injury, illness or other causes. By using the wearable device, which recognises or predicts a child's hunger based on a measured blood glucose level, parents will be able to better recognise their child's needs.

A further advantage may be seen in the fact that a feeling of hunger is recognised early, i.e. already at a time when the feeling has not yet become so strong that the child starts to cry. This may enable the parents to prepare food in good time or, if the parents are travelling with the child, to find a place where the child may be fed in good time.

According to embodiments, the sensors comprise a photoplethysmographic sensor, referred to here as a PPG sensor. The evaluation software is configured to derive the measured blood sugar concentration of the child from the signals detected by the PPG sensor in addition to the heart rate, the oxygen saturation and the breathing rate of the child and to provide at least the blood sugar concentration as input to the evaluation software.

The advantage of this is that the blood sugar concentration may be measured non-invasively and very frequently, e.g. regularly, so that if the blood sugar level drops, the point in time at which the child starts to feel hungry or becomes so hungry that it indicates this by crying may be predicted.

According to some embodiments, the blood parameters used as correction parameters in predicting the increased risk of SIDS are also used to detect erroneous blood sugar measurements.

According to embodiments, the evaluation software is communicatively coupled to an electronic food preparation appliance directly or via a software application of the telecommunication device or via the server-computer system. The evaluation software or telecommunication device software application is configured to activate the electronic appliance in response to the prediction that the feeling of hunger will occur now or in the future, thereby causing the electronic appliance to prepare food for the child.

For example, the electronic appliance may be a milk bottle warmer, a kettle, or a microwave or similar.

### FURTHER EMBODIMENTS

According to embodiments of the invention, the evaluation software may be used to detect and/or predict a variety of physiological states. In addition to predicting an increased risk of sudden infant death syndrome and predicting a feeling of hunger or predicting the time of occurrence of the feeling of hunger, the device may be used, for example, to detect fever and various acute or chronic diseases.

Thus, embodiments of the invention may make it possible to recognise abnormalities which may, for example, be an indication of congenital diseases or acute or chronic diseases.

Thanks to the large number of parameters that may be detected by the wearable device, a very broad database is created that allows a high-quality prediction of the child's current and future physiological states.

According to some embodiments, the evaluation software is configured to selectively recognise the current or future presence of a physiologically problematic state of the child that requires immediate intervention. Such a physiological state may be, for example, an increased risk of sudden infant death syndrome. The evaluation software forwards at least some of the vital parameters or intermediate prediction results measured or derived by the wearable device over a network to the server-computer system without locally calculating a final prediction result to enable the server-computer system to predict physiological states that do not require immediate intervention.

This may be advantageous as the computing capacity of the wearable device is limited due to its small size.

The fact that critical physiological states are predicted by the device itself and non-critical states and/or the detected raw data are sent regularly or in bulk (“bulk upload”) to the server for server-side analysis, e.g. during a charging process, ensures that an alarm signal may always be sent to the telecommunication device by the wearable device itself, regardless of whether or not there is a network connection to the server-computer system. This also ensures that the child may be monitored in every situation—whether sleeping on the sofa in the living room or travelling—with regard to the really critical physiological and physical parameters (SIDS risk, oxygen concentration in the blood, etc.), since the core functionality of the wearable wristband is always available, independently of the server-computer system and independently of the availability of external sensors. The connection to the server-computer system and/or the use of additional parameters provided by external sensors may further refine the prediction result. The prediction quality with regard to critical system states will therefore be somewhat more accurate in the context of the child's usual sleeping area, for example, where the camera, microphone and base station may also be located. Nevertheless, the child may be spontaneously taken to a different location at any time, where neither a network connection nor camera monitoring is possible; the basic protection remains in place as long as the child has the wearable device attached to their body and the caregivers have set up the associated software on the telecommunication device.

The battery of the device is conserved so that the device may be operated for a longer period of time without having to change or recharge the batteries. Processing the prediction results for dispatch requires computing power, for example for converting the data into the correct format for dispatch or for setting up a communication channel. By not sending the measurement results or prediction results for every measurement and every subsequent prediction, but instead only sending the prediction results when a critical or problematic physiological state or parameter value is recognised or predicted, computing power is saved.

In addition, according to some embodiments, the data traffic is reduced because the processing of the raw data relevant for the critical states already takes place on the wearable device, so that, instead of the raw data, only the prediction results and optionally a small amount of raw data relevant for the prediction result need to be transmitted, whereas the entirety of sensor data or at least the sensor data used for the prediction of another physiological state are preferably transmitted collectively at a later time. For example, the data may be transmitted to the telecommunication device via a radio signal, in particular via a radio signal in accordance with the Bluetooth protocol. However, it is also possible for the data to be transmitted via WLAN. According to some embodiments, at least some data is first transmitted from the wearable device to the server-computer system, for example directly via a WLAN connection to the Internet, or indirectly via, for example, radio or WLAN to a base station and from there to the server-computer system.

According to embodiments of the invention, the evaluation software is configured to recognise a current or future presence of a physiologically problematic state of the child,
if a value of at least one vital parameter or environmental parameter is outside a predetermined normal range; and
if a pattern of values of several vital parameters is detected, which indicates a current or future problematic physiological state of the child, wherein the pattern may also be detected if all vital parameters and/or environmental parameters are individually within their normal range.

The evaluation software is configured to send a message regarding the predicted problematic physiological state to the mobile telecommunication device in response to the detection of a current or future physiologically problematic state.

This means that parents are not only warned if, for example, an increased risk of SIDS is detected, but also if, for example, the blood oxygen level has fallen below a minimum value or if the ambient temperature exceeds a predefined maximum value.

According to embodiments of the invention, the wearable device is a bracelet or a strap on the child's ankle or leg. This may be advantageous as the child's freedom of movement is not restricted, the device is able to be attached to these limbs securely, and above all because it is possible to adjust the contact pressure, for example by using an elastic material in the straps or by adjusting a fastener, so that the sensors are in contact with the child's body with a certain minimum pressure, which increases the quality of the measurements.

For example, the strap may have a length configured to be worn on an arm, an ankle or a leg with a circumference of about 7-15 cm (corresponding to the circumference of the corresponding limbs of babies and small children). For example, the strap may be 7.5 to 20 cm long including the fastening mechanism.

According to embodiments, the sensors comprise one or more pressure sensors which are configured to detect the contact pressure of the device on the child's body. The evaluation software is configured to recognise, on the basis of the measured contact pressure, whether the contact pressure is within a predefined permissible contact pressure range within which the one or more sensors for detecting the vital parameters may work correctly and within which the strap will not cause pressure pain in the child. The evaluation software is configured to issue a warning via a signalling element of the device to the user and/or via the interface to the telecommunication device if the measured contact pressure is outside the permissible contact pressure range. In addition or alternatively, the evaluation software is configured to prevent the measurement of vital parameters by the one or more sensors until the contact pressure is once again within the permissible contact pressure range.

This may be advantageous because it is ensured that the sensors are always in sufficient contact with the body to be able to take meaningful measurements. This reduces the number of incorrect predictions and prevents measurement data from being detected that is not meaningful due to a lack of contact with the body.

The wearable device may, for example, emit a warning regarding the absent contact. For example, the warning may be emitted directly via a loudspeaker integrated in the wearable device or via a light source, for example an LED lamp that lights up or flashes. Additionally or alternatively, the wearable device may also send the warning to a software on the wearable telecommunication device or to a base station, so that the warning regarding the missing contact is issued by the telecommunication device and/or the base station.

Due to the fact that the wearable device issues a warning to the user, the user may reposition the device in a suitable position on the child's body.

According to embodiments of the invention, the wearable device is configured to automatically and regularly detect the measured vital parameters and optionally also the at least one environmental parameter and to analyse them using the analysis software. The data may then be collected and uploaded to the server computer and/or transmitted to the telecommunication device, for example via push or pull functionality. For example, the transmission may take place when the wearable device is charged and/or when the caregivers send a request regarding the current data to the wearable device via the telecommunication device.

According to embodiments, the interface for transmitting data to the telecommunication device is an interface for data transmission via a near-field signal, in particular via a radio signal, in particular a Bluetooth interface.

The wearable device is configured to be operable in a low-radiation and a normal-radiation operating state.

The wearable device is configured to operate in the normal operating mode when no physiological state is predicted and in the low-radiation operating mode when no vital or environmental parameter requiring immediate intervention is measured. If the evaluation software detects the current or future presence of a physiologically problematic state, in particular an increased risk of sudden infant death syndrome and/or a feeling of hunger, or the presence of a vital or environmental parameter in a health-critical value range, the wearable device automatically switches to the normal-radiation operating mode. Preferably, the device automatically returns to the low-radiation state after transmitting the relevant data relating to the critical physiological state and/or parameter.

This not only preserves the battery of the wearable device, but also minimises radio radiation, which is considered problematic by some parents.

Depending on the technology used, the low-radiation operating mode may be implemented slightly differently.

When using Bluetooth as a near-field communication technology, switching the wearable device to the low-radiation operating mode may be implemented as follows, for example:

Option 1: Changing the “Advertising Rate” within the Advertising Operating Mode

In this variant, the wearable device works both in the normal operating mode and in the low-radiation operating mode in the so-called “advertising” mode. In this operating mode of Bluetooth devices, the device in question is not permanently connected (paired) with other devices. The wearable device and the telecommunication device are therefore not paired in this operating mode and the data detected by the sensors is not transmitted from the wearable device to the telecommunication device. In the “advertising” state, the wearable device sends out a so-called “advertising” data packet by radio at regular intervals, e.g. 10 seconds or 1 minute, with the information that the wearable device exists, but that it does not wish to establish a connection with the telecommunication device. The rate at which these “advertising” data packets are sent is referred to as the “advertising rate”. A “low-radiation operating state” here is an “advertising” state of a Bluetooth-enabled device in which the advertising rate is below a predefined maximum value, e.g. a maximum of one advertising data packet per minute or a maximum of one advertising data packet per 10 seconds. A “normal-radiation operating state” here is an “advertising” state of a Bluetooth-capable device in which the advertising rate is above the predefined maximum value, e.g. more than one advertising data packet per minute or more than one advertising data packet per 10 seconds.

Within this “advertising” operating mode, the wearable device is normally in the low-radiation operating mode if no critical physiological state is predicted. In this mode, the vital parameters are stored and analysed locally in the wearable device and an advertising data packet is sent at low frequency, which substantially only contains the fact that the wearable device exists but does not wish to be coupled to other devices.

As soon as the evaluation software of the wearable device predicts a critical physiological state requiring immediate intervention, the evaluation software increases the frequency of transmission of the “advertising” data packets so that the data packet may be transmitted as quickly as possible. Preferably, after transmission of one or more data packets containing an alarm and/or measured values relating to the predicted critical physiological state and/or critical vital or environmental parameters, the wearable device switches from the “low-radiation” to the “normal-radiation” operating mode. After sending the alarm data packet(s), the wearable device and its radio module return to the low-radiation operating mode.

All devices within range of this advertising data packet that have already been connected to the wearable device (that were paired with it), in particular all telecommunication devices for which this applies, may receive and process this data packet and, if necessary, show it to the user on the display of the telecommunication device. For greater certainty that the alarm message will reach the telecommunication device, the advertising request may also contain the information that the wearable device now wishes to connect (pair) with the telecommunication device. As soon as this connection is established, the wearable device may also recognise that the data packet with the alarm has reached the recipient.

Option 2: Changing the “Feedback Rate” within a Paired Operating Mode

According to this implementation variant, the wearable device is paired to the telecommunication device of a caregiver in the normal operating mode, i.e. there is an active connection between the wearable device and the telecommunication device. Normally, Bluetooth devices in paired operating mode send very frequent requests (e.g. approx. 100 times per second) to ascertain whether the connected device is still there and expects feedback from the connected device that this is the case.

In the low-radiation operating mode, i.e. when the wearable device does not predict a physiological state requiring intervention or measure environmental or vital parameters requiring intervention, the wearable device operates in a low-radiation operating mode in which the wearable device is coupled to the telecommunication device but reports back to it that it will not report for a predefined number of further feedback messages (e.g. the next 100 feedback messages). This means that fewer feedback data packets are sent from the wearable device to the telecommunication device. If, on the other hand, a critical physiological state or vital parameters or environmental parameters are predicted or measured, the wearable device immediately sends a feedback message with information regarding the state requiring intervention to the paired telecommunication device without waiting for the “cancelled” feedback cycles to expire. The wearable communication device initially switches to the normal-radiation operating mode, as feedback messages are sent at the usual frequency for the paired Bluetooth state. However, as soon as the warning with information regarding the state requiring intervention has been transmitted to the paired telecommunication device, the wearable device automatically returns to the low-radiation operating mode by reporting back that it will not report for a predefined number of further feedback messages. The predefined number of further feedback messages is preferably more than 50, further preferably more than 100 feedback messages.

Option 3: Reducing the Transmission Power when the Connection is Good

According to a third implementation variant, the wearable device is coupled to the telecommunication device in normal operating mode and continuously determines the quality of the connection. For example, it is determined how frequently an expected feedback message is not received or how strong the signal strength is of the telecommunication device's Bluetooth signal.

If no critical system states have currently been predicted and no critical environmental or vital parameters have been detected or calculated, and if the connection quality to the telecommunication device is above a predefined minimum quality level, the wearable device reduces the transmission power of the Bluetooth radio module and thereby enters the low-radiation operating mode. If the connection quality is poor, i.e. below the predefined minimum quality level, or if a physiological state has been predicted or an environmental or vital parameter has been detected that requires immediate intervention, the wireless module of the wearable device maintains or increases the transmission power of the wireless module.

This may save energy and extend the service life of the battery.

In addition to Bluetooth, other standards and/or protocols may also be used for near-field-based data exchange, e.g. ZigBee.

When using Bluetooth or ZigBee, for example, the wearable device has a radio module (“transmission module”). If there are no abnormalities that require the immediate attention of the caregiver, the data is stored locally and the transmission module is operated in a low-radiation mode. In this state, the wearable device and the receiver device (i.e. the wearable telecommunication device and optionally also the base station) are not continuously synchronised according to embodiments. If the evaluation software in the wearable device determines that an abnormality and/or critical physiological state is present, the wireless module is switched to the normal-radiation operating mode and data (measured values and/or prediction results) is sent to the receiver device.

As the range of a Bluetooth signal or ZigBee signal in many households is often shorter than the WLAN signal, for example, WLAN or other suitable Internet-based data communication between the analysis software and the telecommunication device may be implemented as an alternative to Bluetooth or ZigBee.

According to embodiments, the wearable device comprises one or more environmental parameter sensors selected from a group comprising:
a thermometer to measure the ambient temperature;
a measuring device for measuring the ambient air moisture;
gases, especially CO₂;
a microphone for detecting ambient noises and/or noises made by the child;
a UV sensor for detecting a cumulative UV radiation dose, in particular a daily cumulative UV radiation dose.

According to embodiments, the sensors for detecting the vital parameters comprise further sensors selected from a group comprising:
acceleration sensor, e.g. for recognising the position (especially prone or supine position) of the child;
thermometer for measuring the child's skin temperature;
video camera (in particular thermal imaging camera for recognising a prone or supine position).

In a further aspect, the invention relates to a system comprising the device and one or more of the following further components:
the wearable telecommunication device, wherein a user software is set up on the wearable telecommunication device, wherein the user software is interoperable with the evaluation software and is configured to display the prediction results received from the wearable device via the interface to the user and/or to allow the user to configure the evaluation software; and/or
the server-computer system; and/or
a base station to which one or more external sensors for measuring vital parameters of the child or environmental parameters of the child's surroundings are coupled; the base station is configured to forward the parameter values measured by the external sensors to the server system in original or processed form. For example, the external sensors that are or may be communicatively coupled to the base station may comprise a video camera, in particular a thermal imaging camera, a microphone, an ambient temperature sensor, an air moisture sensor and/or a sensor for detecting the CO₂ concentration of the ambient air. According to some embodiments, the base station comprises a module for charging the wearable device, for example via an induction field. According to some embodiments, the base station comprises a network interface, in particular a WIFI interface, for communicatively coupling the base station and/or the external sensors communicatively coupled thereto to the server-computer system. The base station also includes software that is interoperable with the evaluation software on the wearable device as well as with the server application; and/or
one or more of the external sensors, in particular a video camera, in particular a thermal imaging video camera.

According to embodiments of the invention, the predictive software comprises at least one predictive model for predicting the at least one physiological state. The at least one predictive model is a model generated by a machine learning method based on a training dataset. In particular, the predictive model may be a neural network. Neural networks have proven to be particularly suitable for detecting and predicting the relationships between various vital parameters and/or environmental parameters as well as various physiological states.

Predictive models based on machine learning make it possible to recognise complex dependencies between the parameters as well as with the physiological state to be predicted and to take them into account in the prediction. Especially in the field of physiology, vital parameters and environmental parameters often interact in a complex and non-linear way, reinforcing or weakening each other. Machine learning methods, e.g. neural networks, are able to recognise these complex parameter dependencies and use them in the prediction so that not only current physiological states may be predicted, but also states that are likely to occur in the future (and possibly also the time of occurrence).

In a further aspect, the invention relates to a method for providing a wearable device for monitoring the physiological state of a child.

The method comprises a step of providing a training dataset. The training dataset includes several datasets. At least one physiological state of the child is specified in each dataset and is stored in a linked manner with vital parameters of the child (in particular heart rate, oxygen saturation and respiratory rate, possibly also skin temperature or derived core body temperature, movement patterns, video or audio data, etc.). Optionally, the dataset may also contain one or more environmental parameters, e.g. ambient temperature, air moisture, CO₂ concentration of the ambient air, etc. Preferably, the dataset contains a plurality of data values for each of the vital parameters and/or environmental parameters, each of which is stored linked to a time stamp, wherein the physiological state is also stored linked to a time stamp. This makes it possible to recognise not only correlations between several parameters and physiological states, but also their temporal dependencies.

The method further comprises a step of carrying out a machine learning process on the training data to generate the at least one predictive model. The at least one predictive model is configured to predict the current or future physiological state of the child on the basis of at least the heart rate, the oxygen saturation, and the respiratory rate and optionally other vital parameters and/or environmental parameters. According to some embodiments, the predictive model also learns temporal dependencies so that it is also able to predict the time of occurrence of the physiological state for a given set of parameter values.

The method further comprises installing evaluation software, which includes the at least one predictive model, on the wearable device. The device is configured to be worn on a child's body, wherein the device is sized and shaped to be worn by a baby or small child.

The device comprises one or more sensors for detecting several vital parameters of the child. The vital parameters include at least the heart rate, the oxygen saturation and the respiratory rate. The evaluation software is configured to use the at least one predictive model to predict the physiological state on the basis of the measured values detected by the sensors. The wearable device further comprises an interface for transmitting a prediction result relating to the physiological state to a mobile telecommunication device of a user (a caregiver, e.g. a parent) and/or to a server-computer system.

The interface for communication with the telecommunication device is preferably an interface for near-field communication, e.g. by radio or WLAN, but according to some embodiments the interface may also be a mobile radio connection.

The interface for communication with the server-computer system may be a WLAN connection or a mobile phone connection, for example.

According to embodiments of the invention, the at least one model comprises a SIDS model for predicting a current or future increased risk of sudden infant death syndrome. Optionally, the at least one model may comprise one or more further predictive models, e.g. a hunger model for predicting whether and/or when the child will experience a feeling of hunger.

In the training phase, the SIDS model is trained on training data comprising at least the oxygen concentration, the heart rate and the respiratory rate. Preferably, in addition to the oxygen concentration, the training data also includes one or more other blood parameters that serve as control parameters, e.g. CO₂ concentration, methaemoglobin and/or carboxyhaemoglobin. The blood parameters of the training data are preferably detected under real conditions, which means that the training data also contains oxygen concentrations in the blood that are too low due to measurement errors and that are annotated as incorrect in the training data.

The training dataset for training the hunger model preferably contains a large number of datasets, each of which includes a number of other time-stamped parameters in addition to the time at which a feeling of hunger occurs, e.g. the blood sugar level.

In a further aspect, the invention relates to a wearable device configured to be worn on the body of a child, wherein the child is a baby or small child. The device comprises:
at least one sensor for non-invasive detection of the child's blood sugar level as a vital parameter; and
evaluation software configured to predict at least a current or future physiological state of the child in the form of a current or future feeling of hunger in the child as a function of the blood sugar level measured by the sensor; and
an interface for transmitting the prediction result relating to the at least one physiological state to a mobile telecommunication device of a user and/or to a server-computer system.

In a further aspect, the invention relates to a method for providing a wearable device for monitoring a physiological state of a child, wherein the physiological state is a current or future feeling of hunger in the child. The method comprises:
providing a training dataset comprising a plurality of datasets, wherein in each dataset at least one physiological state of the child is stored linked to vital parameters of the child, wherein the vital parameters comprise non-invasively measured blood sugar concentrations of the child;
performing a machine learning process on the training data to generate at least one predictive model, wherein the model is configured to predict the current or future presence of a feeling of hunger in the child on the basis of at least the blood sugar concentration;
installing evaluation software that includes the at least one predictive model on the wearable device, wherein the device is configured to be worn on the body of a child, wherein the child is a baby or small child, wherein the device comprises:
   a sensor for non-invasive detection of the blood sugar concentration, wherein the evaluation software is configured to use the at least one predictive model for predicting the physiological state on the basis of the blood sugar concentration detected by the sensor; and
   an interface for transmitting a prediction result relating to the at least one physiological state to a mobile telecommunication device of a user and/or to a server-computer system.

In a further aspect, the invention relates to a method for providing a wearable device for monitoring a first and a second physiological state of a child, wherein the first physiological state is a state of increased risk of sudden infant death syndrome, and wherein the second physiological state is a current or future feeling of hunger in the child. The method comprises:
providing a first training dataset comprising a plurality of datasets, wherein in each dataset at least the first physiological state of the child is stored linked to vital parameters of the child, wherein the vital parameters comprise at least the heart rate, the oxygen saturation, and the respiratory rate, and providing a second training dataset comprising a plurality of datasets, wherein in each dataset at least the second physiological state of the child is stored linked to vital parameters of the child, wherein the vital parameters comprise non-invasively measured blood sugar concentrations of the child;
performing a machine learning process on the first and second training data to generate at least a first and a second predictive model, wherein the first predictive model is configured to predict the first physiological state of the child based on at least the heart rate, the oxygen saturation, and the respiratory rate, wherein the second predictive model is configured to predict the second physiological state of the child on the basis of at least the blood sugar concentrations;
installing evaluation software comprising the at least one predictive model on the wearable device, wherein the device is configured to be worn on the body of a child, wherein the child is a baby or small child, wherein the evaluation software is configured to use at least the heart rate, the oxygen saturation, and the respiratory rate as input to predict the presence of an increased risk of sudden infant death syndrome, wherein the evaluation software is configured to use at least the blood sugar concentrations as input to predict a current or future feeling of hunger in the child as a function of at least the measured blood sugar concentration, and/or to predict a future time of occurrence of the feeling of hunger, wherein the feeling of hunger is predicted when the current or future blood sugar level is below a predefined threshold value, wherein the device comprises:
   one or more sensors for detecting a plurality of vital parameters of the child, wherein the vital parameters comprise at least the heart rate, the oxygen saturation, the respiratory rate, and the blood sugar concentration, wherein the evaluation software is configured to use the first predictive model to predict the first physiological state on the basis of the heart rate, oxygen saturation and respiratory rate detected by the sensors and is configured to use the second predictive model to predict the second physiological state on the basis of the blood sugar concentration detected by the sensors; and
   an interface for transmitting a prediction result relating to the first and second physiological states to a mobile telecommunication device of a user and/or to a server-computer system.

A “wearable device” is defined here as an electronic device that is worn on the user's body during use. They are also referred to as “wearables”. For example, the device may be attached to the body or integrated into clothing using certain fastening means (e.g. strap, in particular hook-and-loop fastener, buckle fastener, magnetic fastener, etc.). Preferably, the device comprises one or more sensors and a data processing unit.

A “telecommunication device” is understood here to mean any portable data processing device capable of data transmission via a network, in particular a mobile phone, smartphone, smartwatch, tablet computer or notebook.

The term “child” is used here to refer to a small child or baby. A “small child” here means a child in the second, third or fourth year of life; a “baby” means here a child in the first year of life.

The term “battery” is used here to refer to a non-rechargeable primary battery or a rechargeable secondary battery (commonly known as an accumulator).

A “predictive model” is understood here to be an executable file, a parameter set and/or a data structure that enables a software program, or is itself configured, to predict the current presence of a specific physical state of an entity and/or to predict the future existence of this state. Typically, a predictive model uses historical data relating to the state to be recognised or predicted for the calculation. For example, the historical data may be used as training data in order to extract the knowledge contained in this data in the course of a machine learning process and store it in the predictive model. In particular, the knowledge may include knowledge of parameter correlations.

The expression “machine learning” is understood here to mean a process by which knowledge about the relationships between several parameters contained in training data is transferred into a so-called “model”, which may be used to automatically calculate predictions regarding the properties of entities and processes. This means that the examples are not simply learnt by heart, but patterns and regularities are recognised in the learning data. In this way, the system may also assess unknown data (learning transfer). For example, the generated model may be a predictive model in the form of a trained artificial neural network or other data structures such as support vector machines.

A “vital parameter” is understood here to be a data value, in particular a numerical value, which reflects a state and/or a current property of a person's body. A vital parameter may be a data value or raw data value that is obtained directly as a measured value through a measurement, or a value derived by calculation from measured raw data.

An “environmental parameter” is understood here to mean a data value, in particular a numerical value, which is completely or at least largely dependent on entities outside a person's body. For example, the strength of the sun's UV radiation is an environmental parameter, as is the room temperature, since a certain amount of heating of a room by a person's body heat is possible, but the effect is generally negligible. An ambient parameter value may be a data value or raw data value, which is obtained directly as a measured value by a measurement, or a value derived by calculation from measured raw data.

The term “physiological state” is used here to describe a biophysical state of certain life processes of an organism, e.g. a child. The state may be a healthy state, a pathological state or a risky state, for example. For example, a state in which all vital parameters are within the normal range is generally regarded as healthy, and a state in which one or more important biophysical parameters deviate from the normal range and cause current complaints is described as a pathological state. A “risky” state is one in which the person's health is not currently impaired, but in which the risk of a pathological state occurring is significantly increased.

### BRIEF DESCRIPTION OF THE DRAWING

Embodiments of the invention are described below with reference to the drawing. The drawing shows

FIG. 1 a method for providing a wearable device for predicting a physiological state of a child;

FIG. 2 an illustration of a variant of the device configured as a wristband;

FIG. 3 an illustration of the transmission of an alarm via near-field signal from the wristband to a smartphone;

FIG. 4 a block diagram of a region of a wearable device with a plurality of sensors;

FIG. 5 a diagram showing the derivation and use of various parameters for the prediction of an increased risk of SIDS;

FIG. 6 a diagram showing the derivation and use of a parameter for the prediction of feelings of hunger;

FIG. 7 a system for monitoring the health of a child comprising a plurality of components.

FIG. 1 shows methods for providing a wearable device for predicting a physiological state of a child.

In a first step 102, a training dataset is provided. For example, the training dataset may be provided on a storage medium or downloaded via a network.

For example, the training dataset may have been generated by attaching a wearable device comprising a plurality of sensors to multiple small children and babies to capture and store multiple vital parameters and/or environmental parameters over an extended period of time. In addition, the data thus obtained is annotated with verified physiological states. If the number of children is sufficiently large and the observation period sufficiently long, various, sometimes critical situations and corresponding physiological states will occur. For example, colds and associated fevers may occur. Feelings of hunger may occur in the short term if there are delays while travelling and it is not possible to adhere to the child's feeding times. Abnormal breathing patterns (e.g. apnoea), hypoxaemia and bradycardia may also occur. These and other abnormalities may occur in premature babies in particular and may be detected and saved as a training dataset. The generation of the training dataset may also provide for external sensors to be used in addition to the wearable device in order to detect additional vital parameters and/or environmental parameters so as to increase the size of the training dataset.

In the next step 104 a predictive model is trained using a machine learning method. Various methods such as neural networks, support vector machines and similar methods may be used. However, neural networks have proven to be particularly advantageous in this context. The entirety of the parameters and their respective time stamps represent the input parameters of the model to be trained. The annotated physiological states of the child represent the output data. In the course of training, various parameters of the model, for example weights of neural network nodes, are adjusted so that the output (physiological state) predicted by the model on the basis of a set of input parameters is, to the greatest possible extent, identical or similar to the physiological states that were actually observed and annotated in the training dataset. This process may involve minimising a so-called “loss function”.

In a further step 106, the trained predictive model may be integrated into evaluation software and this may be installed on one or more wearable devices and/or the server-computer system. A software application interoperable with the evaluation software may be provided as a so-called “app” via the app store of the respective operating system provider of the telecommunication device for download and installation on the telecommunication device.

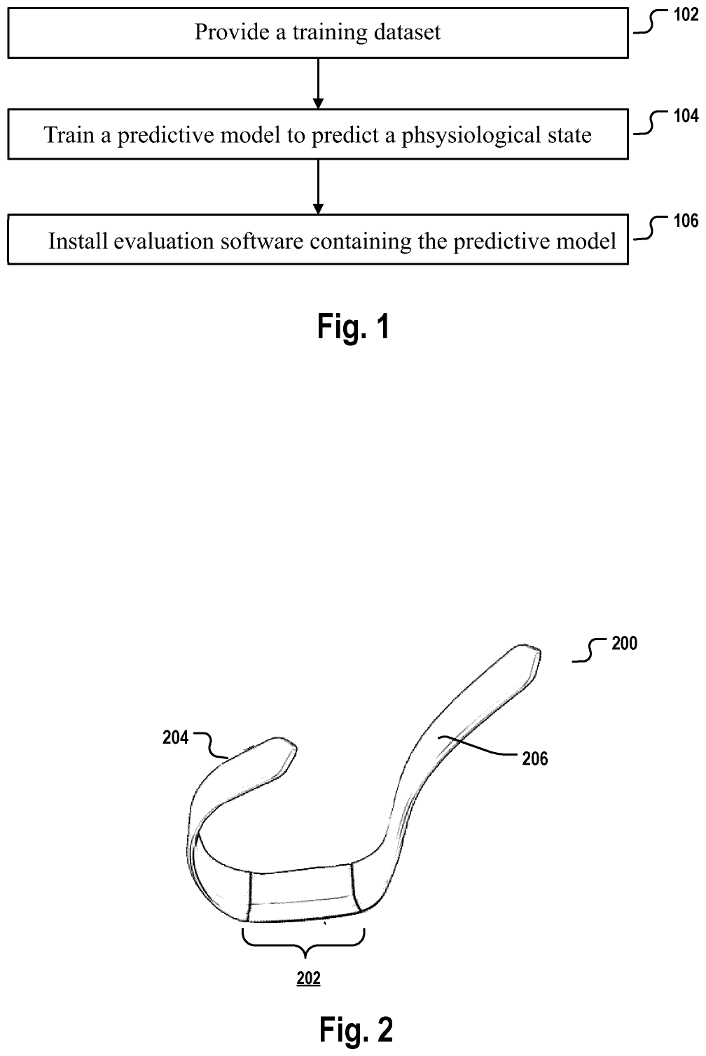

FIG. 2 shows an illustration of a variant of the wearable device 200 formed as a wristband. In the variant shown here, all or the majority of the sensors are located within a central sensor block 202 to which two arms 204, 206 are attached. The size, shape and material of the device are configured to be worn on the wrists, ankles, arms or legs of a baby or small child. For example, the arms may be made of a flexible material such as silicone or fabric. They have a locking mechanism that allows the device 200 to be securely attached to the child's body. Preferably, the material of the arms 204, 206 is elastic in order to ensure sufficient contact pressure.

In other embodiments, however, the sensors may also be distributed over one of the two arms or over both arms.

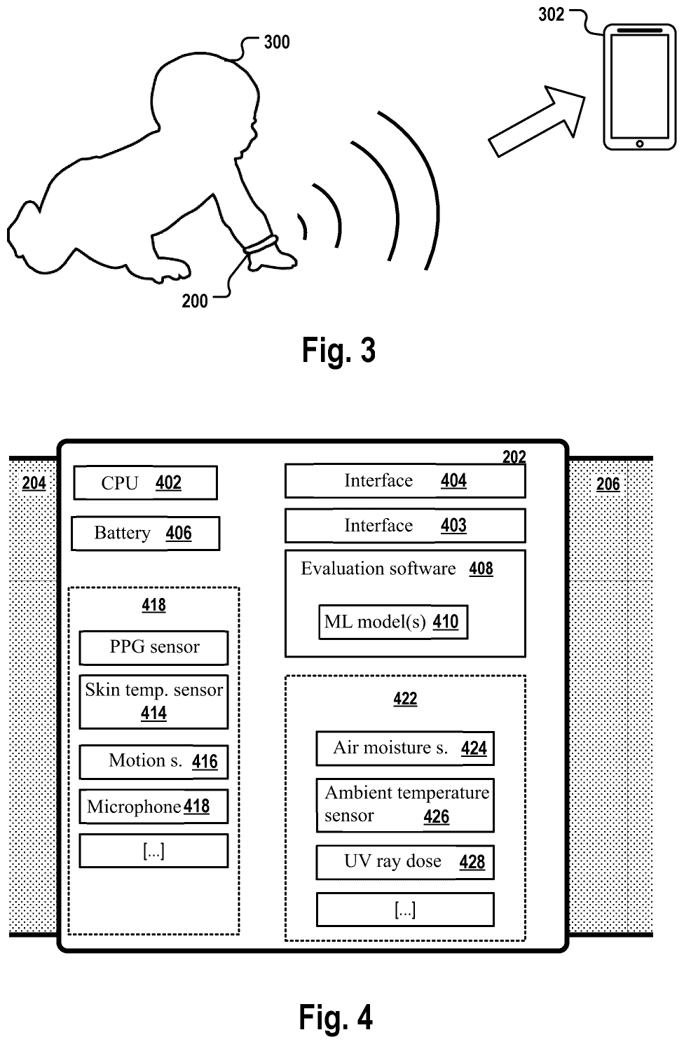

FIG. 3 shows an illustration of the transmission of an alarm via near-field signal from the wristband to a smartphone. For example, the wristband 200 may have a module for near-field communication, for example a Bluetooth module. The radio signal of the Bluetooth standard is generally sufficient to penetrate one or two walls, so that a wearable telecommunication device 302, for example a smartphone of the parent, may receive warning signals from the wearable device 200 even if the parent is, for example, briefly in another room but still in close proximity to the child. The telecommunication device 302 may include software that generates an output on the basis of the data received from the wearable device 200 (in particular, prediction results regarding physiological states, but optionally also raw data or vital parameters derived from the raw data). For example, the output may comprise a GUI that is shown on the display of the smartphone and contains, for example, a warning regarding the predicted physiological state and/or a recommendation for action. The recommendation may, for example, consist of turning the child over, giving them food, lowering or raising the temperature in the room or similar. In addition or as an alternative to the visual output via the display, the caregiver may also be warned acoustically, for example by an alarm sound or by activating the vibration function of the smartphone.

FIG. 4 shows a block diagram of a region of a wearable device comprising a plurality of sensors. The wearable device 200 includes a battery 406 and one or more processors 402, which may be, for example, microprocessors.

The device includes an interface 404 for exchanging data with the wearable telecommunication device 302, for example a radio interface. Preferably, it also includes an interface 403 for exchanging data with a server-computer system. The interface 403 may, for example, be a mobile radio connection or a WLAN connection in order to be able to exchange data with the server-computer system via the Internet.

Evaluation software 408 is installed on the wearable device. The software may include one or more predictive models 410, each of which has been trained, for example, to predict a particular physiological state (for example, increased risk of sudden infant death syndrome, occurrence of a feeling of hunger, occurrence of fever, etc.). However, rule-based algorithms may also be used instead of the models.

The sensor module 202 includes one or more sensors 418 for sensing vital parameters. In particular, the module 202 includes a PPG sensor 412, from the raw data of which a variety of relevant vital parameters may be derived, including, for example, heart rate, respiratory rate, blood oxygen concentration, blood glucose concentration, and several other vital parameters or blood components serving as controls in SIDS prediction. In some embodiments, the device 200 includes other vital parameters sensors, such as a skin temperature sensor 414, a gyroscope 416 for detecting movement of the child, and/or a microphone 418.

In addition, the sensor module 202 may include other sensors 422 for detecting environmental parameters, for example, an air moisture sensor 424, an ambient temperature sensor 426, and/or a daily or hourly UV radiation dose sensor 428. For example, the sensor 428 may be used to detect the UV light dose to which the child has been exposed during the course of a day. If the recommended maximum dose is reached or exceeded, the evaluation software may send a warning to the smartphone app that the child must be protected from further sun exposure. However, detecting the daily UV light dose over a period of time may also help to identify an undersupply of sunlight.

Depending on the embodiment, various sensors from different manufacturers may be used, some of which differ in the way they process the detected measurement data. For example, temperature sensors usually indicate the temperature in degrees Celsius or degrees Fahrenheit. A PPG sensor signal 112, on the other hand, provides one or more light spectra, whereby one or more vital parameters such as the oxygen concentration of the blood or the glucose concentration are only obtained by subsequent processing of the spectra.

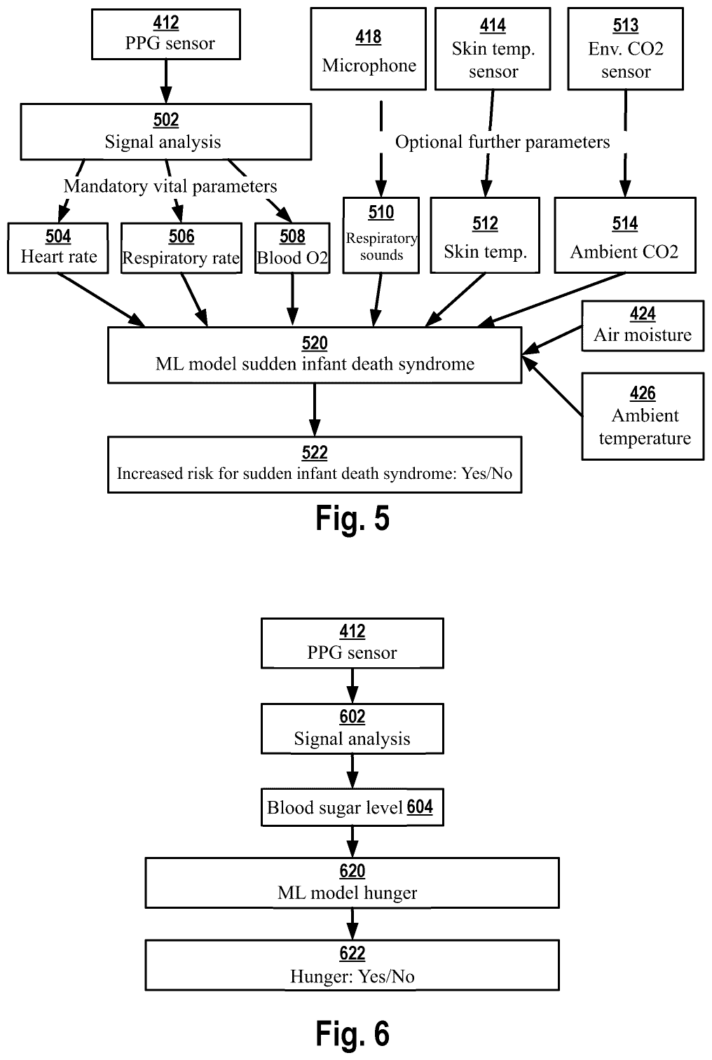

FIG. 5 shows a diagram relating to the derivation and use of various parameters for predicting an increased risk of SIDS. For example, the evaluation software on the wearable device 200 may include a SIDS model 520 that requires at least the heart rate 504, the respiratory rate 506 and the oxygen concentration in the blood of the child 508 as input parameters. These vital parameters may be obtained, for example, by a signal analysis 502 based on the raw data or spectral data from a PPG sensor 412. The stated three vital parameters are always available as long as the child is only wearing the wearable device on the body. The SIDS model 520 is also configured to analyse other input parameters in order to make the prediction of an increased risk of sudden infant death syndrome even more accurate. These include, for example, some control parameters in the form of blood parameter values, which may also be derived from the raw data of the PPG sensor, for example (not shown here). In addition, this may include other vital parameters, such as skin temperature 512 detected by a temperature sensor 414 of the device, breath sounds 510 detected by a microphone 418, carbon dioxide concentration in the ambient air detected by a carbon dioxide sensor 513, air moisture 424 and/or ambient temperature 426. Analysing the breath sounds may, for example, be used to determine whether breathing is affected by obstruction, which would imply an increased risk of SIDS. Movement data and/or video data (not shown here) may also be included in the prediction, as they may indicate, for example, whether the child is active or whether the child is lying on its stomach or back.

If the prediction shows that the child is currently or will soon be at an increased risk of sudden infant death syndrome, the device 200 sends an alarm message 522 either directly to the caregiver's smartphone or indirectly to the server computer, where the prediction result may be further refined if necessary using data provided by external sensors via the base station. The refined prediction result is then forwarded by the server-computer system via the network to the caregiver's smartphone and output there, provided that the refined prediction result also indicates an increased risk of sudden infant death syndrome.

FIG. 6 shows a diagram relating to the derivation and use of a parameter for the prediction of feelings of hunger. Here, too, the PPG sensor 412 may first be used to detect one or more light spectra that are reflected by the child's skin and the vessels in the skin. From the raw data, the evaluation software uses signal analysis 602 to determine at least one derived vital parameter, namely the blood sugar level 604. At least the blood sugar level and optionally other parameters are used as input parameters in the prediction of a current or future feeling of hunger by a trained predictive “hunger” model 620. The result 622 of the prediction as to whether and, if so, when a feeling of hunger is present or will be present is either sent directly to the smartphone via a near-field signal or indirectly via the server-computer system, wherein the server-computer system is used in particular to refine the prediction result by including further data from other sensors and/or by additional, possibly computationally complex analyses.

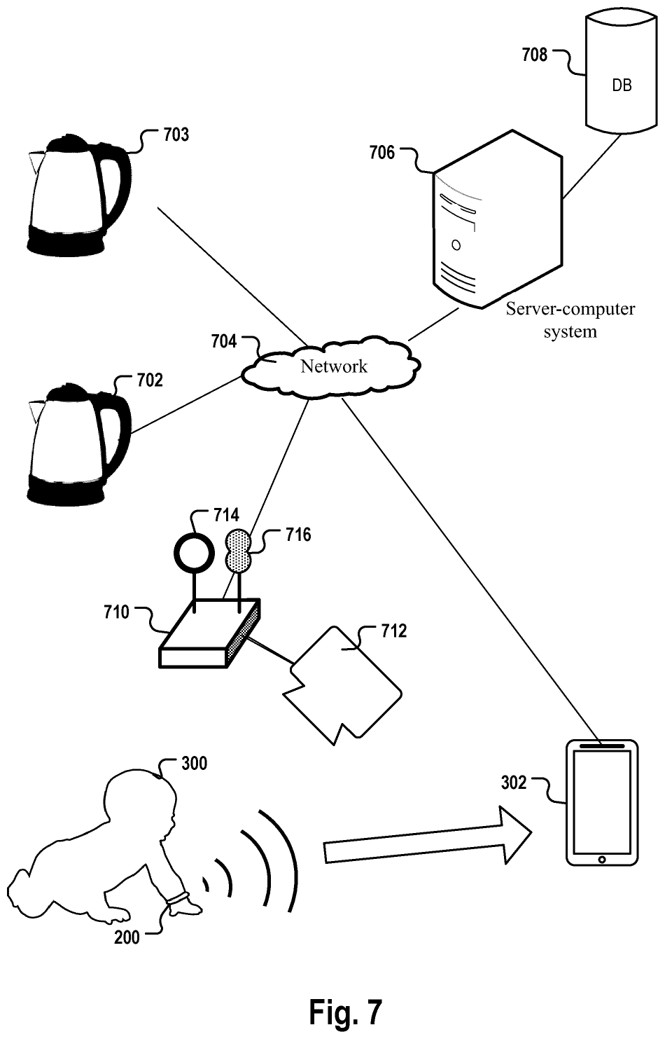

FIG. 7 shows a system for monitoring the health of a child comprising a plurality of components. The system comprises at least the wearable device 200, which is configured here, for example, as a bracelet for attachment to the wrist of a child 300.

The system may also include one or more wearable telecommunication devices 302, typically smartphones of the caregivers, on which software is installed that is interoperable with the evaluation software of the device 200 in order to be able to exchange data therewith. For example, the owners of the telecommunication device 302 may be notified of critical physiological states of the child via push message from the evaluation software and/or may actively request status data or historical data regarding the physiological states of the child 300 from the wearable device 200 via pull functionality.

The system may further include a server-computer system 706 that is connected to the wearable device 200 and the evaluation software 408 via a network 704, such as the Internet. For example, depending on the urgency and configuration, the data collected and possibly derived by the device 200 as well as prediction results may be transmitted to the server-computer system via the network immediately or, for example, during the battery charging process. In particular, the server-computer system is used to store the received data from one or more devices 200 or external sensors 712 in a database 708. In addition, prediction results received via the network 704 from the wearable device 200 and its sensors may be refined and made more precise on the server-computer system. In particular, this may be done by additionally taking into account additional data determined by external sensors 712 and transmitted directly to the server-computer system via the network (e.g., the Internet) or indirectly via a base station 710 and/or by the server-computer system performing complex, computationally elaborate analyses. For example, a microphone 716 and/or a camera 712 (in particular a thermal imaging camera) or other additional sensors 714 may be installed as external sensors in or on the bed in which the child normally sleeps. These external sensors are communicatively coupled to the server-computer system 706 either directly via the network or indirectly via a base station 710 and may send data thereto. For example, a server application on the server-computer system may perform an image analysis of the video data from the camera 712, for example to recognise whether the child is in the supine or prone position, which is an important prognostic factor for the risk of sudden infant death syndrome.

According to one embodiment, the external sensor is a video camera, in particular a thermal imaging video camera, which is communicatively connected to the wearable telecommunication device via an interface for near-field communication (e.g. radio, in particular Bluetooth, or WLAN) in order to enable the caregivers to monitor the baby by video signal. Preferably, the video camera is wearable and may be set up freely and may be communicatively coupled to the server computer, e.g. via WLAN over the Internet, preferably even without a base station. This may have the advantage that the parents may also install the camera in the vicinity of their child without major installation effort, e.g. when they are travelling, so that the mobility of the parents is increased.

According to one variant, the evaluation software of the device 200 and/or the application on the smartphone 302 that is interoperable with this evaluation software is operatively coupled via the network 704 to one or more appliances 702, 703 that are used to prepare or cook food for the child. The appliances 702, 703 may be, for example, a microwave oven, a kettle, an appliance for heating milk or baby food, etc. If the wearable device 200 recognises or predicts by means of the evaluation software that the child is currently hungry or will be hungry in the near future, the evaluation software may automatically send a control command to one or more of the appliances 702, 703 to cause it to start preparing food. Preferably, however, the control command is not sent directly to said appliances 702, 703, but first to the software on the smartphone 302. In response to receiving the control command, the smartphone software prompts the user to authorise sending the control command to the appliance in question. The smartphone then sends the control command to the relevant appliance 702, 703 after receiving the user's approval. This ensures that the evaluation software does not automatically activate an appliance remotely without the caregivers knowing about it, as this could pose a security risk.

The server-computer system 706 may be a conventional, monolithic server computer. However, it may also be a distributed server architecture, in particular a cloud computer system.

1. A wearable device,
wherein the wearable device is configured to be worn on the body of a child, wherein the child is a baby or small child;
wherein the device comprises:
one or more sensors for sensing a plurality of vital parameters of the child, wherein the vital parameters comprise at least heart rate, oxygen saturation, and respiratory rate; and
evaluation software configured to predict at least a current or future physiological state of the child as a function of the heart rate, oxygen saturation, and respiratory rate measured by the sensors; and
an interface for transmitting the prediction result relating to the at least one physiological state to a mobile telecommunication device of a user and/or to a server-computer system.

2. The wearable device according to claim 1,
wherein the at least one physiological state is a state of increased risk of sudden infant death syndrome,
wherein the evaluation software is configured to use at least the heart rate, oxygen saturation, and respiratory rate as input to predict the presence of an increased risk of sudden infant death syndrome.

3. The wearable device according to claim 2, wherein the evaluation software is configured to predict the presence of an increased risk of sudden infant death syndrome as a function of one or more further parameters, wherein the one or more further parameters comprise:
the child's skin temperature;
the ambient temperature; and/or
the ambient air moisture.

4. The device according to claim 1,
wherein the sensors comprise a photoplethysmographic sensor, referred to here as a PPG sensor,
wherein the evaluation software is configured to derive the heart rate, oxygen saturation and respiratory rate of the child from the signals detected by the PPG sensor and to make them available as input to the evaluation software.

5. The device according to claim 1,
wherein the one or more sensors comprises a sensor for detecting at least one blood parameter of the child, wherein the at least one blood parameter is in particular a methemoglobin concentration and/or a carboxyhemoglobin concentration and/or a CO₂ concentration in the blood of the child, wherein the sensor for detecting the blood parameter may be configured in particular as the PPG sensor according to claim 4;
wherein the evaluation software is configured to use the at least one blood parameter as an additional input parameter in order to reduce the false positive rate of the prediction of the increased risk of sudden infant death syndrome by the evaluation software.

6. The device according to claim 1,
wherein the device comprises at least one sensor for determining at least one further vital parameter and/or environmental parameter, or an interface for receiving the further vital parameter and/or environmental parameter from an external sensor, wherein the at least one further environmental parameter is selected from a group comprising:
the CO₂ concentration of the ambient air,
video data of the child, in particular video data of an infrared camera;
acoustic data detected by a microphone; and
movement data characterising the child's movement activity;
wherein the evaluation software is configured to use the at least one further vital parameter and/or environmental parameter as an additional input parameter in order to predict the presence of an increased risk of sudden infant death syndrome.

7. The wearable device according to claim 1,
wherein at least one of the sensors for the vital parameters is configured to measure the child's blood sugar concentration in a non-invasive manner;
wherein the evaluation software is configured to predict a further physiological state in the form of a current or future feeling of hunger in the child as a function of at least the measured blood sugar concentration, and/or to predict a future time of occurrence of the feeling of hunger.

8. The device according to claim 7,
wherein the sensors comprise a photoplethysmographic sensor, referred to here as a PPG sensor,
wherein the evaluation software is configured to derive the child's blood sugar concentration from the signals detected by the PPG sensor in addition to the heart rate, the oxygen saturation and the breathing rate of the child and to provide at least the blood sugar concentration as input at least for predicting the feeling of hunger.

9. The device according to claim 1,
wherein the evaluation software is configured to recognise the current or future presence of a physiologically problematic state of the child,
when a value of at least one vital parameter is outside a predetermined normal range; and/or
when a pattern of values of multiple vital parameters is detected that indicates a current or future problematic physiological state of the child, wherein the pattern may be detected even if all vital parameters are individually within their normal range; and
wherein the evaluation software is configured to send a message regarding the predicted problematic physiological state to the mobile telecommunication device and/or the server-computer system in response to the detection of the current or future physiologically problematic state.

10. The device according to claim 1,
wherein the evaluation software is configured to selectively recognise the current or future presence of a physiologically problematic state of the child requiring immediate intervention, wherein this physiologically problematic state comprises in particular the increased risk of sudden infant death syndrome; and
forwarding at least some of the vital parameters or intermediate prediction results measured or derived by the wearable device to the server-computer system via a network to enable the server-computer system to predict physiological states and/or calculate a refined final prediction, in particular of physiological states that do not require immediate intervention.

11. The device according to claim 7,
wherein the evaluation software is communicatively coupled to an electronic food preparation appliance directly or by means of a software application of the telecommunication device; and
wherein the evaluation software and/or the software application of the telecommunication device is configured to cause the electronic appliance to prepare food for the child in response to the prediction that the child is feeling hungry now or will do in the future.

12. The device according to claim 1, wherein the device is a bracelet or ankle or leg strap.

13. The device according to claim 12,
wherein the sensors comprise one or more pressure sensors which are configured to detect the contact pressure of the device on the child's body,
wherein the evaluation software is configured to recognise, on the basis of the measured contact pressure, whether the contact pressure is within a predefined permissible contact pressure range within which the one or more sensors for detecting the vital parameters are able to operate correctly,
wherein the evaluation software is configured to issue a warning via a signalling element of the device to the user and/or via the interface to the telecommunication device if the measured contact pressure is outside the permissible contact pressure range; and/or
wherein the evaluation software is configured to prevent the measurement of vital parameters by the one or more sensors until the contact pressure is again within the permissible contact pressure range.

14. The device according to claim 1, wherein the device is configured
to only send a message to the user device if the evaluation software has detected the current or future presence of a physiologically problematic state, in particular an increased risk of sudden infant death syndrome and/or a feeling of hunger, or the presence of a vital or environmental parameter in a health-critical value range; and
otherwise to automatically save the detected vital parameters and optionally detected environmental parameters without sending a message.

15. The device according to claim 1,
wherein the interface for transmitting data to the telecommunication device is an interface for data transmission via a near-field signal, in particular via a radio signal, in particular a Bluetooth interface or ZigBee interface,
wherein the wearable device is operable in a low-radiation operating mode and in a normal-radiation operating mode;
wherein the wearable device, in the normal operating mode, is configured to operate in the low-radiation operating state when no physiological state is predicted and no vital or environmental parameter requiring immediate intervention is measured; and wherein the wearable device is configured to automatically switch to the radiation-normal operating mode if the evaluation software has detected the current or future presence of a physiologically problematic state, in particular an increased risk of sudden infant death syndrome and/or a feeling of hunger, or the presence of a vital or environmental parameter in a health-critical value range.

16. The wearable device according to claim 1,
wherein the wearable device comprises one or more environmental parameter sensors selected from a group including:
a thermometer that measures the ambient temperature;
a measuring device for measuring the ambient air moisture;
gases, especially CO₂;
UV sensor for detecting a cumulative UV radiation dose, in particular a daily cumulative UV radiation dose;
and/or wherein the sensors of the wearable device for detecting the vital parameters comprise further sensors selected from a group comprising:
acceleration sensor for detecting the position of the child;
temperature sensor for detecting the skin temperature;
a microphone for detecting ambient noises and/or noises made by the child;
video camera, in particular a thermal imaging camera.

17. The wearable device according to claim 1, wherein the predictive software comprises at least one predictive model for predicting the at least one physiological state, wherein the at least one predictive model is a model generated by a machine learning method on the basis of a training dataset.

18. A system comprising the wearable device according to claim 1, and one or more of the following further components:
the portable telecommunication device, wherein a user software is set up on the portable telecommunication device, wherein the user software is interoperable with the evaluation software and is configured to display the prediction results received from the wearable device via the interface to the user and/or to enable the user to configure the evaluation software; and/or
the server-computer system; and/or
a base station to which one or more external sensors are coupled for measuring vital parameters of the child or environmental parameters of the child's surroundings; and/or
one or more of the external sensors, in particular a video camera, in particular a thermal imaging video camera.

19. A method for providing a wearable device for monitoring the physiological state of a child, comprising:
providing a training dataset comprising a plurality of datasets, wherein in each dataset at least one physiological state of the child is stored linked to vital parameters of the child, wherein the vital parameters comprise at least heart rate, oxygen saturation, and respiratory rate;
performing a machine learning process on the training data to generate at least one predictive model, wherein the model is configured to predict the physiological state of the child on the basis of at least heart rate, oxygen saturation, and respiratory rate;
installing evaluation software including the at least one predictive model on the wearable device, wherein the device is configured to be worn on the body of a child, wherein the child is a baby or small child, wherein the device comprises:
one or more sensors for detecting a plurality of vital parameters of the child, wherein the vital parameters comprise at least heart rate, oxygen saturation, and respiratory rate, wherein the evaluation software is configured to use the at least one predictive model to predict the physiological state on the basis of the heart rate, oxygen saturation, and respiratory rate detected by the sensors; and
an interface for transmitting a prediction result relating to the at least one physiological state to a mobile telecommunication device of a user and/or to a server-computer system.
